(19) United States
(12) Patent Application Publication
(10) Pub. No.: US 2017/0296490 A1
(43) Pub. Date: Oct. 19, 2017

(54) USE OF LIGNIN-DERIVED ALDEHYDES AS ANTIFUNGAL AGENTS

(71) Applicant: Wisconsin Alumni Research Foundation, Madison, WI (US)

(72) Inventors: Jeff S. Piotrowski, Madison, WI (US); Steve D. Karlen, Verona, WI (US); Medhi Kabbage, Sun Prairie, WI (US); Ashish Ranjan, Madison, WI (US)

(21) Appl. No.: 15/489,545

(22) Filed: Apr. 17, 2017

Related U.S. Application Data
(60) Provisional application No. 62/323,059, filed on Apr. 15, 2016.

(51) Int. Cl. A61K 31/085; A61K 9/00; A01N 31/16; A61K 31/05; A01N 31/08

(52) U.S. Cl. CPC A61K 31/085; A61K 31/05; A01N 31/08; A01N 31/16; A61K 9/0014

(57) ABSTRACT

Described herein is a method and corresponding composition of matter for inhibiting the growth of fungi and oomycetes. The method includes contacting fungi or oomycetes with a growth inhibitory effective amount of one or more compounds selected from p-hydroxycoumaryl aldehyde, coniferyl aldehyde, and sinapaldehyde, and salts and esters thereof.

### CROSS-REFERENCE TO RELATED APPLICATIONS

Priority is hereby claimed to provisional application Ser. No. 62/323,059, filed Apr. 15, 2016, which is incorporated herein by reference.

### FEDERAL FUNDING STATEMENT

This invention was made with government support under DE-FC02-07ER64494 awarded by the US Department of Energy. The government has certain rights in the invention.

### BACKGROUND

Fungal pathogens represent one of the greatest economic threats to sustainable crop production. Fungal pathogens also present significant health risks to humans and animals. In North America, the fungal pathogen landscape is changing as a warming climate brings novel pathogens from Central and South America. Because fungal pathogens evolve resistance rapidly, new antifungal compounds for treating crops, humans, and animals are in increasing demand.

Very serious damage is done to crops and trees each year by fungal pathogens that run the gamut of the fungal world: smuts, rusts, ergots, mildews, etc. Dutch elm disease, for example, is caused by the fungus Ceratostomella ulmi. First seen in the United States in the late 1920's, by 1989 Dutch elm disease had wiped out over 75% of the elm trees in the United States.

Other fungal pathogens are more promiscuous. Alternaria alternata, for example, is a fungus which has been recorded as causing leaf spot and other diseases on over 380 host species of plant. It is an opportunistic pathogen on numerous hosts causing leaf spots, rots, and blights on many plant parts. In humans, it can also cause upper respiratory tract infections and asthma in subjects with compromised immunity. Similarly, the diploid fungus Candida albicans causes a number of opportunistic oral and genital infections in humans. Systemic fungal infections caused by C. albicans are prevalent in immunocompromised patients.

### SUMMARY

These compounds represents a new class of antifungal agents for both plant and animal pathogens. Further, given their significantly inhibition of many pathogens in the unmodified forms, they could be rapidly developed for this market. Most importantly is that it is a natural product that could qualify as a new fungicide that may be compliant with organic agriculture standards. Currently, the primary treatment for plant pathogens like Sclerotinia is the synthetic carboxamide trivially named Boscalid (3-pyridinecarboxamide, 2-chloro-N-(4′chloro[1,1′biphenyl]-2-yl). Oomycete pathogens such as Phytophthora are treated with the synthetic fungicide trivially named Metalaxyl (2-[(2,6-dimethylphenyl)-(2-methoxy-1-oxoethyl) amino]propanoic acid methyl ester). Organic treatments for fungi are largely limited to broadly acting metals formulations such as copper sulfate. As all 3 agents have described activity against known human and plant pathogens, they already have a large potential market. Further testing may reveal activity against a broader set of pathogens.

The Ascomycete Sclerotinia sclerotiorum is a plant pathogenic fungus that causes huge crop loses throughout the world.

Also provided are pharmaceutical compositions (e.g., single unit dosage forms) that can be used in the methods provided herein. In one embodiment, pharmaceutical compositions comprise a compound provided herein, or a pharmaceutically acceptable form (e.g., salts, hydrates, solvates, isomers, prodrugs, and isotopically labeled derivatives) thereof, and optionally one or more additional active agents.

While specific embodiments have been discussed, the specification is illustrative only and not restrictive. Many variations of this disclosure will become apparent to those skilled in the art upon review of this specification.

Unless defined otherwise, all technical and scientific terms used herein have the same meaning as is commonly understood by one of skill in the art to which this specification pertains.

As used in the specification and claims, the singular form “a”, “an,” and “the” includes plural references unless the context clearly dictates otherwise.

The term “effective amount” refers to that amount of a compound or pharmaceutical composition described herein that is sufficient to effect the intended application including, but not limited to, disease treatment or inhibition, as illustrated below. The therapeutically effective amount can vary depending upon the intended application (in vitro or in vivo), or the subject and disease condition being treated, e.g., the crop being treated, the weight and age of the subject, the severity of the disease condition, the manner of administration and the like, which can readily be determined by one of ordinary skill in the art. The term also applies to a dose that will induce a particular response in target cells, e.g., reduction of fungal growth or cell count. The specific dose will vary depending on, for example, the particular compounds chosen, the dosing regimen to be followed, whether it is administered in combination with other agents, timing of administration, the tissue or plant to which it is administered, and the physical delivery system in which it is carried.

As used herein, the terms “treatment” or “treating” refer to an approach for obtaining beneficial or desired results including, but not limited to, therapeutic benefit and/or a prophylactic benefit. By therapeutic benefit is meant eradication or amelioration of the underlying fungal infection being treated. Also, a therapeutic benefit is achieved with the eradication or amelioration of one or more of the physiological symptoms associated with the underlying disorder such that an improvement is observed in the patient or plants, notwithstanding that they can still be afflicted with the underlying fungal infection. For prophylactic benefit, the pharmaceutical compositions can be administered to a patient at risk of developing a particular fungal infection, or to a patient reporting one or more of the physiological symptoms of a fungal infection, even though a diagnosis of a fungal infection may not have been made.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods of the present invention can comprise, consist of, or consist essentially of the essential elements and limitations of the method described herein, as well as any additional or optional ingredients, components, or limitations described herein or otherwise useful in agronomic, botanical, or pharmaceutical chemistry.

### BRIEF DESCRIPTION OF THE DRAWINGS

FIG. 1 is a series of photographs illustrating dose-dependent inhibition of Alternaria solani by sinapaldehyde (top row) and coniferyl aldehyde (bottom row) at concentrations of 250 μg/mL, 500 μg/mL, and 1,000 μg/mL.

FIG. 2 is a graph depicting dose-dependent inhibition of Alternaria alternata by sinapyl aldehyde. Control=. 250 μg/mL=∘. 500 μg/mL=▾. 1,000 μg/mL=Δ.

FIG. 3 is a graph depicting dose-dependent inhibition of Alternaria alternata by coniferyl aldehyde. Control=. 250 μg/mL=∘. 500 μg/mL=▾. 1,000 μg/mL=Δ.

FIG. 4 is a series of photographs illustrating dose-dependent inhibition of Scelerotinia sclerotiorium by sinapyl aldehyde (top row) and coniferyl aldehyde (bottom row) at concentrations of 250 μg/mL, 500 μg/mL, and 1,000 μg/mL.

FIG. 5 is a graph depicting dose-dependent inhibition of the yeast S. cerevisiae by sinapyl aldehyde (), coniferyl aldehyde (∘), and p-hydroxycoumaryl aldehyde (▾).

FIG. 6 is a graph depicting dose-dependent inhibition of the yeast C. albicans by sinapyl aldehyde (), coniferyl aldehyde (∘), and p-hydroxycoumaryl aldehyde (▾).

### DETAILED DESCRIPTION

Identified herein are new set of products within the lignin biosynthesis pathway the exhibit pronounced antifungal activity. These compounds, p-hydroxycoumaryl aldehyde, coniferyl aldehyde, and sinapyl aldehyde, show inhibition of not only the plant pathogens Alternaria solani, Alternaria alternata, and Sclerotinia sclerotiorium, but also the yeast Saccharomyces cerevisiae and the human pathogen Candida albicans.

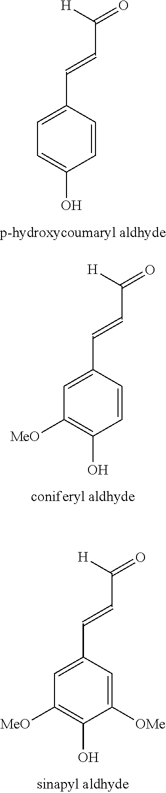 [SMILES: O=C/C=C/c1ccc(O)cc1 ; COc1cc(/C=C/C=O)ccc1O ; COc1cc(/C=C/C=O)cc(OC)c1O]

Additionally, these agents are very strong inhibiters of the oomycete pathogen Phytophthora, displaying a complete inhibition of growth at 250 ug/mL. Chemical genomics profiling suggest these compounds affect central cytoskeleton dependent processes. S. sclerotiorum alone is estimated to cause $250 million annually in crop damage in the United States alone. When considered with pathogenic Alternaria, Phytophthora, and Candida, the total economic costs of these pathogens is over $1 billion annually. These aldehydes from the lignin biosynthesis pathway are thus a new class of natural fungicides with significant economic potential.

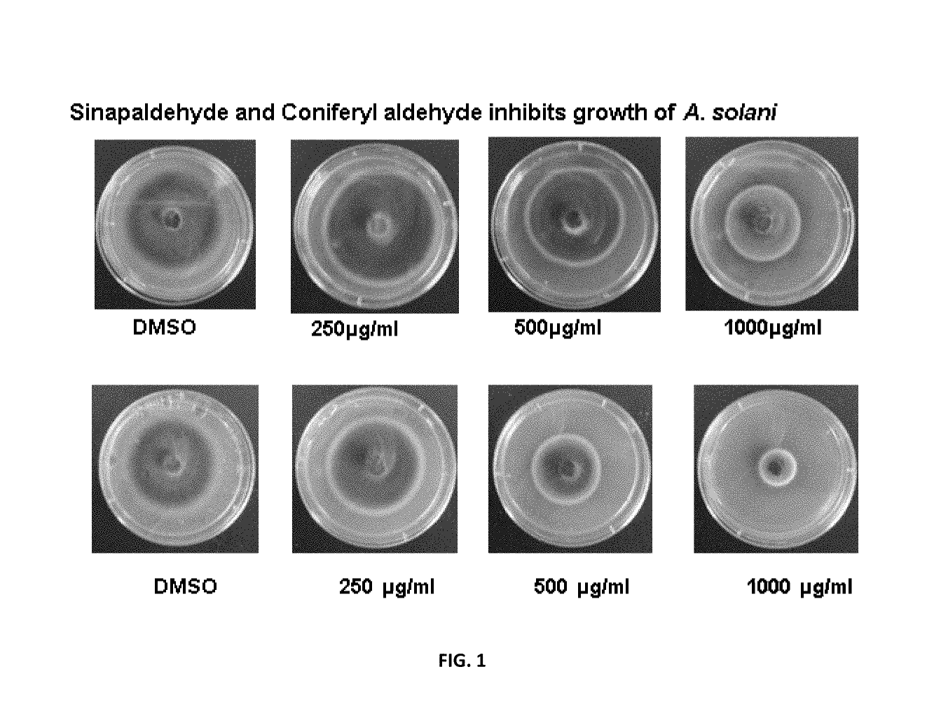

As shown in FIG. 1, both sinapaldehyde and coniferyl aldehyde inhibit growth of A. solani in a dose-dependent fashion. The top row of photos in FIG. 1 shows growth of a colony of A. solani in an untreated control (DMSO), and in the presence of 250 μg/mL, 500 μg/mL, and 1,000 μg/mL of sinapaldehyde. As can be seen the top row of photos, growth of the colony was very significantly inhibited as the dose of sinapaldehyde increased. Likewise, the bottom row of photos in FIG. 1 shows growth of a colony of A. solani in an untreated control (DMSO), and in the presence of 250 μg/mL, 500 μg/mL, and 1,000 μg/mL of coniferyl aldehyde. As can be seen the bottom row of photos, growth of the colony was very significantly inhibited as the dose of coniferyl aldehyde increased. These results are significant because A. solani is the fungal pathogen responsible for early blight in potatoes, tomatoes, and other nightshades. Without aggressive early control of the pathogen, early blight causes significant yield reductions. FIG. 1 demonstrates that the propagation and spread of A. solani can be inhibited by applying sinapaldehyde and/or coniferyl aldehyde to the potentially affected plants.

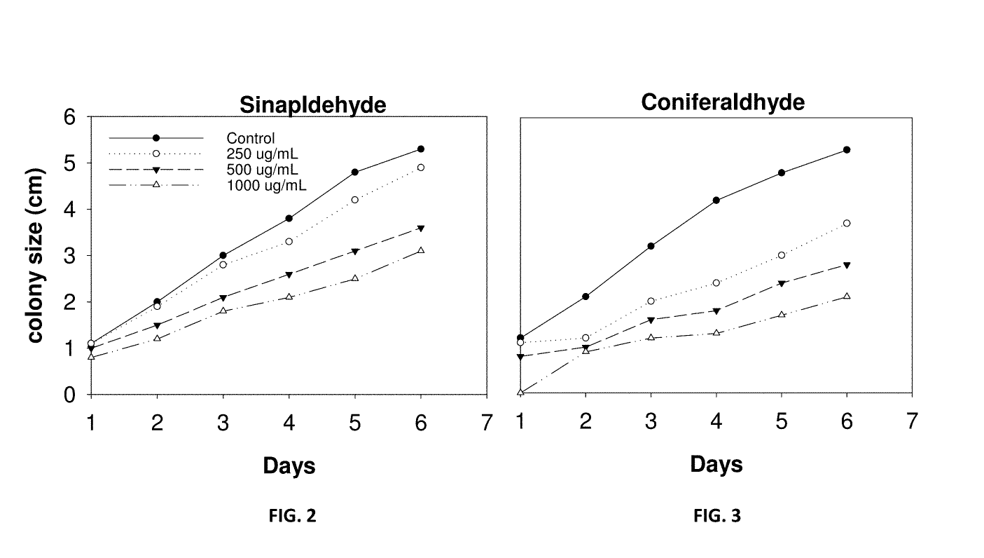

Similar to FIG. 1, FIG. 2 is a graph depicting dose-dependent inhibition of Alternaria alternata by sinapyl aldehyde. Time in days is on the X-axis; colony size in cm is on the Y-axis. (Control=. 250 μg/mL=∘. 500 μg/mL=▾. 1,000 μg/mL=Δ.) As can be seen from FIG. 2, the untreated control group has the most rapid increase in colony size and the largest colony size after the 6-day course of the experiment. The treatment groups each had progressively attenuated growth rates and smaller colony size at the end of six days. In short, the colony treated with 250 μg/mL sinapyl aldehyde (∘) was smaller than the untreated colony; the colony treated with 500 μg/mL sinapyl aldehyde (▾) smaller still; and the colony treated with 1,000 μg/mL sinapyl aldehyde (Δ) the smallest of those tested. These results are significant because A. alternata causes leaf spot and other diseases on over 380 host species of plant. A. alternata has also been implicated as a causative agent of asthma in humans. Salo et al. (October 2006) “Exposure to Alternaria alternata in US homes is associated with asthma symptoms” J Allergy Clin Immunol.; 118(4): 892-898. FIG. 2 thus demonstrates that sinapaldehyde is active to inhibit the growth of A. alternate, a fungal species that is economically significant do to its impact on agriculture and a human pathogen as well.

FIG. 3 is a graph depicting the dose-dependent inhibition of Alternaria alternata by coniferyl aldehyde In the same fashion as in FIG. 2, the treatment groups each had progressively attenuated growth rates and smaller colony size at the end of six days. (Control=. 250 μg/mL=∘. 500 μg/mL=▾. 1,000 μg/mL=Δ.)

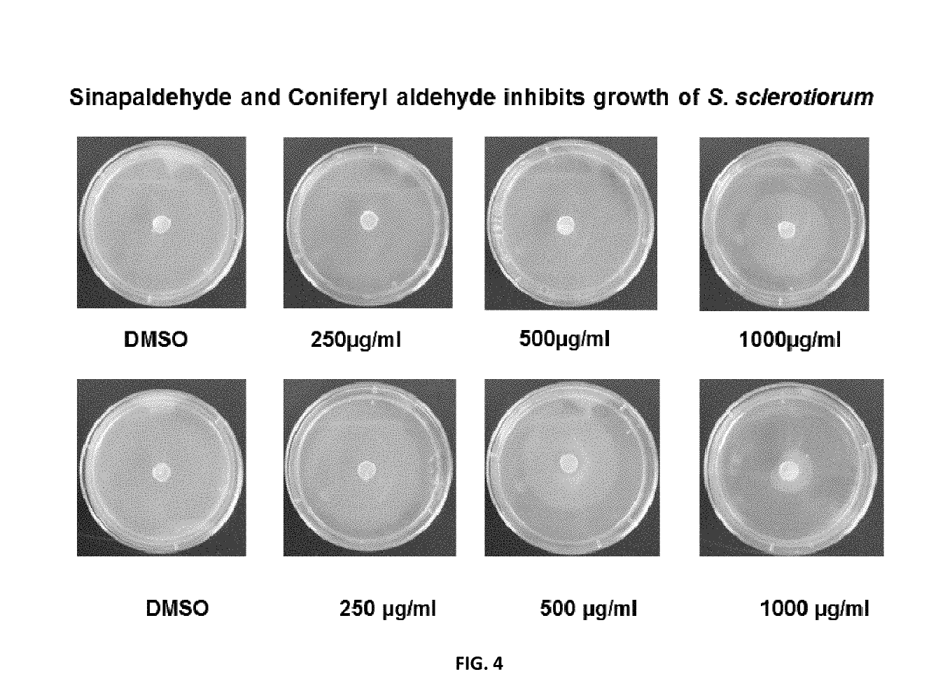

FIG. 4 is a series of photographs analogous to FIG. 1, but showing the ability of sinapldehyde and coniferyl aldehyde to attenuate the grown of S. sclerotiorum. As shown in FIG. 4, both sinapaldehyde and coniferyl aldehyde inhibit growth of S. sclerotiorum in a dose-dependent fashion. The top row of photos in FIG. 4 shows growth of a colony of S. sclerotiorum in an untreated control (DMSO), and in the presence of 250 μg/mL, 500 μg/mL, and 1,000 μg/mL of sinapaldehyde. As can be seen the top row of photos, growth of the colony was very significantly inhibited as the dose of sinapaldehyde increased. Likewise, the bottom row of photos in FIG. 4 shows growth of a colony of S. sclerotiorum in an untreated control (DMSO), and in the presence of 250 μg/mL, 500 μg/mL, and 1,000 μg/mL of coniferyl aldehyde. As can be seen the bottom row of photos, growth of the colony was very significantly inhibited as the dose of coniferyl aldehyde increased. These results are significant because S. sclerotiorum, when conditions are appropriate, causes a fungal plant disease commonly known as white mold. White mold is particularly significant economically because it affects a wide range of hosts. It is also capable of infecting plants at any stage of growth makes. S. sclerotiorum, can survive on infected tissues, in the soil, and on living plants. It affects young seedlings, mature plants, and fruit in the field or in storage. Notably, under humid, warm conditions, S. sclerotiorum spreads very quickly from plant to plant and can infect and entire field in just days. It can also propagate throughout a harvested crop if conditions are not monitored carefully. It infects many extensively planted crops, including soybeans, green beans, sunflowers, canola, and peanuts. FIG. 4 demonstrates that the propagation and spread of S. sclerotiorum can be inhibited by applying sinapaldehyde and/or coniferyl aldehyde to the potentially affected plants, to the soil of a field, etc.

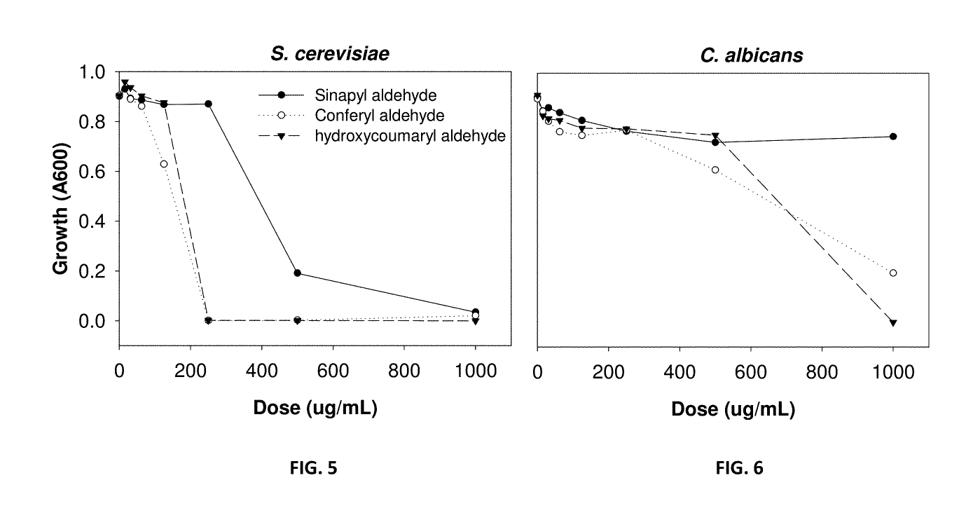

FIGS. 5 and 6 are graphs demonstrating the ability of sinapyl aldehyde, coniferyl aldehyde, and hydroxycoumaryl aldehyde to inhibit the growth of two very common organisms, namely the yeast Saccharomyces cerevisiae, widely used in baking, brewing and winemaking, and the pathogenic diploid fungus Candida albicans.

FIG. 5 depicts dose-dependent inhibition of S. cerevisiae by sinapyl aldehyde (), coniferyl aldehyde (∘), and p-hydroxycoumaryl aldehyde (▾). All three compounds exhibited very significant inhibition at 500 μg/mL. Coniferyl aldehyde and hydroxycoumaryl aldehyde displayed complete inhibition of the growth of S. cerevisiae at concentrations well below 300 μg/mL

FIG. 6 depicts dose-dependent inhibition of C. albicans by sinapyl aldehyde (), coniferyl aldehyde (∘), and p-hydroxycoumaryl aldehyde (▾). Here, the inhibition was not as pronounced as against S. cerevisiae. Nevertheless, coniferyl aldehyde (∘), and p-hydroxycoumaryl aldehyde (▾) displayed very signification inhibitory activity at a concentration of 1000 μg/mL.

Taken together, FIGS. 5 and 6 clearly demonstrate that the subject compounds are effective to inhibit the growth of S. cerevisiae and C. albicans.

The compounds disclosed herein, p-hydroxycoumaryl aldehyde, coniferyl aldehyde, and sinapaldehyde (also called sinaplyl aldehyde), are all aldehydes that include a hydroxyl group. The compounds are thus amendable to being made into salts. The compounds can be used in their free hydroxyl form, or in the form of salts thereof, preferably salts that are tolerated by a plant or animal substrate to which the salts are to be applied. As a general rule, the salts can be any acid or base addition salt whose counter-ions are non-toxic to the substrate to which the salt is applied (at the relevant doses of the salts), so that the beneficial inhibitory effects inherent in the free base or free acid are not vitiated by side-effects ascribable to the counter-ions. A host of suitable salts are well known in the art. For basic active ingredients, all acid addition salts are useful as sources of the free base form even if the particular salt, per se, is desired only as an intermediate product as, for example, when the salt is formed only for purposes of purification, and identification, or when it is used as intermediate in preparing a pharmaceutically-suitable salt by ion exchange procedures. These salts include, by way of example and without limitation, those derived from mineral acids and organic acids, explicitly including hydrohalides, e.g., hydrochlorides and hydrobromides, sulphates, phosphates, nitrates, sulphamates, acetates, citrates, lactates, tartrates, malonates, oxalates, salicylates, propionates, succinates, fumarates, maleates, methylene bis b hydroxynaphthoates, gentisates, isethionates, di p toluoyltartrates, methane sulphonates, ethanesulphonates, benzenesulphonates, p toluenesulphonates, cyclohexylsulphamates, quinates, and the like. Base addition salts include those derived from alkali or alkaline earth metal bases or conventional organic bases, such as triethylamine, pyridine, piperidine, morpholine, N-methylmorpholine, and the like. See, for example, “Handbook of Pharmaceutical Salts, Properties, Selection, and Use, Second Edition,” P. H. Stahl and C. G. Wermuch, Eds., © 2011, Wiley-VCH (Zurich, Switzerland), ISBN: 978-3906390512.

In addition to the lignin-derived aldehydes note above, the antifungal compositions disclosed herein may optionally contain one or more additional antifungal compounds, such as enilconazole (also known as imazalil; (RS)-1-[2-(Allyloxy)-2-(2,4-dichlorophenyl)ethyl]-1H-imidazole) (Janssen Pharmaceutica, a wholly owned subsidiary of Johnson & Johson, Raritan, N.J.), thiabendazole (which is found in several commercial products, including “TECTO”® Flowable SC, marketed in the United States by Syngenta Crop Protection, LLC, Greensboro, N.C.), benomyl, captan (a nonsystemic phthalimide fungicide), bitertanol (as found in the commercial product BAYCOR®, Bayer CropScience, LP, Research Triangle Park, N.C.), prochloraz (N-propyl-N-[2-(2,4,6-trichlorophenoxy)ethyl]imidazole-1-carboxamide) and formalin and commercial products marketed under the names “TOPSIN”® M (Cerexagri B. V., Vondelingenplaat, Netherlands; active ingredient thiofanaat-methyl), “JET”-5® (Certis Europe BV, Maarssen, The Netherlands; active ingredients peracetic acid and hydrogenperoxide), and “SHIRLAN”® (Syngenta, Basel, Switzerland; active ingredient fluazinam).

The composition may contain any number of accessory ingredients, such as excipients and/or drying agents to prevent agglomeration of the product prior to use, and/or sticking agents to improve adherence of the antifungal compound/composition to a plant surface or mucosal surface being treated, e.g., plant leaves, the mucosal lining of the mouth, nose, anus/rectum, vagina, etc.

When applied to crops and other plants, examples of such sticking agents are latex-based products, such as like “PROLONG”®-brand sticking agent (Holland Fyto BV, Emmeloord, The Netherlands) and “BOND”®-brand sticking agent (Loveland Industries Ltd., Greeley, Colo.), pinolene/terpene-based products, such as “NU-FILM”®-brand agent (Hygrotech; Pretoria, South Africa) and “SPRAY-FAST”®-brand agent (Mandops; Hampshire, UK) and long chain polysaccharides like xanthan gum, gellan gum and guar gum. Alternatively, the sticking agent may be a polymer or co-polymer from a type of polymer such as polyacrylate and polyethylene e.g. “NEOCRYL”® (DSM; Heerlen, The Netherlands).

For treating objects with a hydrophobic surface, the composition may contain one or more surfactants. Examples of useful surfactants are anionic tensides such as sodium lauryl sulphate or polyethylene alkyl ethers or polyoxyethylethers, e.g. “TWEEN”®-brand 60, 61 or 65 surfactants (Croda Americas LLC, Wilmington, Del.). Other examples of useful surfactants are organo silicones, sulfosuccinates, alcohol ethoxylates, fatty acid ethoxylates, fatty acid propoxylates and the like.

Wetting agents for applying the composition to mucus membranes, include, but are not limited to, glycerine, sorbitol, xylitol, and the like.

The composition may further (and optionally) comprise additional compounds such as suitable carriers and adjuvants ordinarily employed in formulation technology, including, but not limited to, mineral substances, solvents, dispersants, emulsifiers, wetting agents, stabilizers, preservatives, antifoaming agents, buffering agents, UV-absorbers and antioxidants.

To improve the effectiveness and the practical use of the compositions, additional active ingredients, such as compounds to combat insects, nematodes, mites, and bacteria may be added to the antifungal composition.

The compositions disclosed herein preferably have a pH of from 4 to 8, preferably of from 5 to 7. They may be solid or liquid. Advantageously, they are liquids which can be applied by spraying using conventional equipment.

The compounds disclosed herein and their pharmaceutically acceptable salts possess antifungal activity and can be used to control and/or prevent fungal infections. This is illustrated by the figures and accompanying text contained herein.

In addition to the fungal species specifically disclosed herein, infections caused by other fungi and oomycetes, such as, for example, Aspergillus, Rhizopus, Dermatophytes and Histoplasma spp., among others, may also be effectively treated.

The subject disclosure thus provides a method of treating a host animal or plant having a susceptible fungal infection which comprises administering to the animal or plant an antifungal-effective amount of a compound selected from the group consisting of p-hydroxycoumaryl aldehyde, coniferyl aldehyde, sinapaldehyde, and pharmaceutically acceptable salts thereof. The disclosure also provides pharmaceutical compositions and agricultural compositions comprising an antifungal-effective amount of a compound selected from the group consisting of p-hydroxycoumaryl aldehyde, coniferyl aldehyde, sinapaldehyde, and pharmaceutically acceptable salts thereof and a pharmaceutically or agriculturally acceptable carrier.

As indicated above, the current disclosure also entails pharmaceutical compositions comprising a compound selected from the group consisting of p-hydroxycoumaryl aldehyde, coniferyl aldehyde, sinapaldehyde, and pharmaceutically acceptable salts thereof, together with a pharmaceutically acceptable carrier therefor and, optionally, other therapeutically active substances. The pharmaceutical compositions comprise an amount of a compound selected from the group consisting of p-hydroxycoumaryl aldehyde, coniferyl aldehyde, sinapaldehyde, and pharmaceutically acceptable salts thereof that is effective to inhibit the growth of a fungal infection in an animal suffering therefrom to which the composition is administered. The carrier must be pharmaceutically acceptable in the sense of being compatible with other ingredients in the particular composition and not deleterious to the recipient thereof. The compositions include those suitable for oral, topical, rectal, vaginal, or parenteral (including subcutaneous, intramuscular, intradermal and intravenous) administration.

In a particular aspect, the pharmaceutical compositions comprise the active ingredient (a compound selected from the group consisting of p-hydroxycoumaryl aldehyde, coniferyl aldehyde, sinapaldehyde, and pharmaceutically acceptable salts thereof) presented in unit dosage form. The term “unit dosage” or “unit dose” is defined to mean a predetermined amount of the active ingredient sufficient to be effective for treating each of the indicated activities. Preferred unit dosage formulations are those containing a daily dose, daily sub-dose, or an appropriate fraction thereof, of the administered active ingredient.

The pharmaceutical compositions may be prepared by any of the methods well known in the art of pharmacy. All methods include the step of bringing the active ingredient into association with a carrier which constitutes one or more accessory ingredients. In general, the compositions are prepared by uniformly and intimately bringing the active ingredient into association with a liquid or solid carrier and then, if necessary, shaping the product into the desired unit dosage form.

Compositions of the present invention suitable for oral administration may be presented as discrete unit dosages, e.g., as capsules, cachets, tablets, boluses, lozenges and the like, each containing a predetermined amount of the active ingredient; as a powder or granules; or in liquid form, e.g., as a collyrium, suspension, solution, syrup, elixir, emulsion, dispersion and the like.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients or excipients, e.g., binders, lubricants, inert diluents, surface active or dispersing agents. Molded tablets may be made by molding in a suitable machine, a mixture of the powdered active compound with any suitable carrier.

Compositions suitable for parenteral administration conveniently comprise a sterile injectable preparation of the active ingredient in, for example, a solution which is preferably isotonic with the blood of the recipient. Useful formulations also comprise concentrated solutions or solids containing the active ingredient which upon dilution with an appropriate solvent give a solution suitable for parenteral administration. The parenteral compositions include aqueous and non-aqueous formulations which may contain conventional adjuvants such as buffers, bacteriostats, sugars, thickening agents and the like. The compositions may be presented in unit dose or multi-dose containers, for example, sealed ampules and vials.

Compositions suitable for topical or local application (including ophthamological administration) comprise the active ingredient formulated into pharmaceutically-acceptable topical vehicles by conventional methodologies. Common formulations include drops, collyriums, aerosol sprays, lotions, gels, ointments, plasters, shampoos, transferosomes, liposomes and the like.

Compositions suitable for inhalation administration, for example, for treating fungal infection in the lungs and airways, wherein the carrier is a solid, include a micronized powder or liquid formulation having a particle size in the range of from about 5 microns or less to about 500 microns, for rapid inhalation through the nasal or oral passage from a conventional inhalation squeeze or spray container. Suitable liquid nasal compositions include conventional nasal sprays, nasal drops and the like, of aqueous solutions of the active ingredient and optional adjuvants.

In addition to the aforementioned ingredients, the compositions of this invention may further include one or more optional accessory ingredients(s) utilized in the art of pharmaceutical formulations, e.g., diluents, buffers, flavoring agents, colorants, binders, surfactants, thickeners, lubricants, suspending agents, preservatives (including antioxidants) and the like.

The amount of active ingredient required to be effective for each of the indicated activities will, of course, vary with the individual mammal or plant being treated and is ultimately at the discretion of the medical, veterinary, or agronomy practitioner. The factors to be considered include the species and sex of the animal being treated (or crop being treated), the fungal infection being treated, the route of administration, the nature of the formulation, the mammal's body weight, surface area, age and general condition, and the particular compound to be administered.

In topical formulations, the subject compounds are preferably utilized at concentrations of from about 0.1% to about 20% by weight.

What is claimed is:

1. A method of inhibiting growth of fungi and oomycetes, the method comprising contacting a fungus or an oomycete with a growth inhibitory-effective amount of a compound selected from the group consisting of: 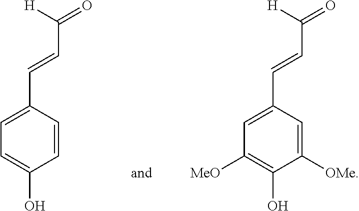 [SMILES: O=C/C=C/c1ccc(O)cc1 ; "and" ; COc1cc(/C=C/C=O)cc(OC)c1O]
and salts and esters thereof.

2. The method of claim 1, wherein the compound is:

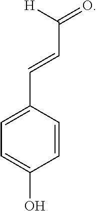 [SMILES: O=C/C=C/c1ccc(O)cc1]

3. The method of claim 1, wherein the compound is:

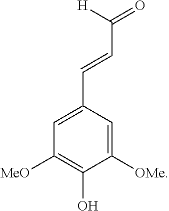 [SMILES: COc1cc(/C=C/C=O)cc(OC)c1O]

4. A method of inhibiting growth of fungi and oomycetes on a plant, the method comprising contacting a plant with a fungus or oomycete growth inhibitory-effective amount of a compound selected from the group consisting of: 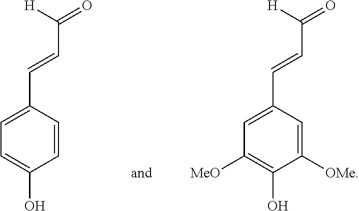 [SMILES: O=C/C=C/c1ccc(O)cc1 ; "and" ; COc1cc(/C=C/C=O)cc(OC)c1O]
and salts and esters thereof.

5. The method of claim 4, wherein the compound is:

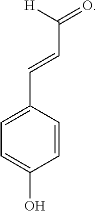 [SMILES: O=C/C=C/c1ccc(O)cc1]

6. The method of claim 4, wherein the compound is:

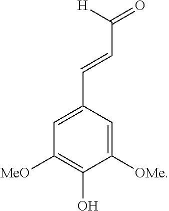 [SMILES: COc1cc(/C=C/C=O)cc(OC)c1O]

7. A composition of matter comprising: a fungi or oomycete growth inhibitory-effective amount of a compound selected from the group consisting of: 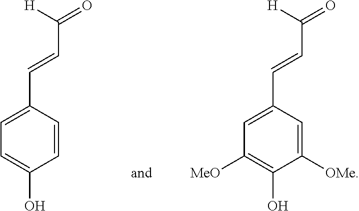 [SMILES: O=C/C=C/c1ccc(O)cc1 ; "and" ; COc1cc(/C=C/C=O)cc(OC)c1O]
and salts and esters thereof, in combination with a carrier suitable for topical administration.

8. The composition of matter of claim 7, wherein the compound is:

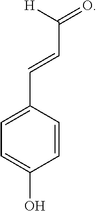 [SMILES: O=C/C=C/c1ccc(O)cc1]

9. The composition of matter of claim 7, wherein the compound is:

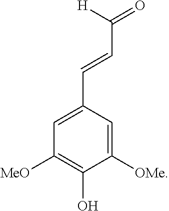 [SMILES: COc1cc(/C=C/C=O)cc(OC)c1O]

10. The composition of matter of claim 7, wherein the carrier is suitable for topical administration to plants.

11. The composition of matter of claim 7, wherein the carrier is suitable for topical administration to mammalian skin.

12. The composition of matter of claim 7, wherein the carrier is suitable for topical administration to mucus membranes.

13. A pharmaceutical composition for inhibiting growth of fungal and or oomycete infections in mammals, the composition comprising a fungi or oomycete growth inhibitory-effective amount of a compound selected from the group consisting of: 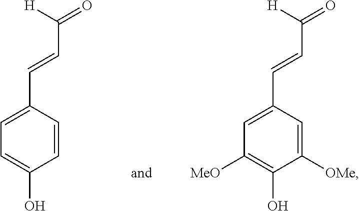 [SMILES: O=C/C=C/c1ccc(O)cc1 ; "and" ; COc1cc(/C=C/C=O)cc(OC)c1O]
and pharmaceutically suitable salts and esters thereof, in combination with a pharmaceutically suitable carrier.

14. The composition of matter of claim 13, wherein the compound is:

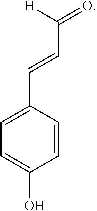 [SMILES: O=C/C=C/c1ccc(O)cc1]

15. The composition of matter of claim 13, wherein the compound is:

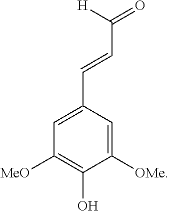 [SMILES: COc1cc(/C=C/C=O)cc(OC)c1O]

16. The composition of matter of claim 13, wherein the carrier is suitable for topical administration to mammalian skin.

17. The composition of matter of claim 13, wherein the carrier is suitable for topical administration to mucus membranes.
